(19) United States
(12) Patent Application Publication
(10) Pub. No.: US 2024/0293186 A1
(43) Pub. Date: Sep. 5, 2024

(54) A SYSTEM AND METHOD OF MERGING A CO-OPERATIVE MR-COMPATIBLE ROBOT AND A LOW-FIELD PORTABLE MRI SYSTEM

(71) Applicant: Neuro42 Inc., San Francisco, CA (US)

(72) Inventors: Hamidreza Hoshyarmanesh, San Francisco, CA (US); Nio Anderson, San Francisco, CA (US); Guanhao Fu, Richmond, CA (US); Abhita Batra, Rocklin, CA (US); Amit Vohra, Rocklin, CA (US); Nikhil Sadhwani, Scottsdale, AZ (US)

(73) Assignee: Neuro42 Inc., San Francisco, CA (US)

(21) Appl. No.: 18/594,625

(22) Filed: Mar. 4, 2024

Related U.S. Application Data
(60) Provisional application No. 63/488,102, filed on Mar. 2, 2023.

(51) Int. Cl. A61B 34/20; A61B 5/00; A61B 5/055; A61B 34/37

(52) U.S. Cl. CPC A61B 34/20; A61B 5/0042; A61B 5/055; A61B 5/704; A61B 34/37; A61B 2034/2051

(57) ABSTRACT

A system can include a magnetic resonance imaging (MRI) scanner and a surgical robot. The MRI scanner can include a permanent magnet array define a dome to surround an imaging region, or region of interest. The MRI scanner can be configured to generate an image of an anatomical structure (e.g. the head of a patient) positioned within the dome and imaging region thereof. The MRI scanner can include an opening for accessing the head of the patient. The surgical robot can include robotic arm. The surgical robot can be mounted to the MRI scanner. The surgical robot can be configured to pass through the opening to perform a surgical procedure on the patient.

### CROSS REFERENCE TO RELATED APPLICATION

The present application claims priority under 35 U.S.C. § 119(e) from U.S. Provisional Application No. 63/488,102, entitled A METHOD OF MERGING A CO-OPERATIVE MR-COMPATIBLE ROBOT AND A LOW-FIELD PORTABLE MRI SYSTEM filed on Mar. 2, 2023, which is incorporated by reference herein in its entirety.

### BACKGROUND

The present disclosure relates to magnetic resonance imaging (MRI), medical imaging, medical intervention, and surgical intervention. MRI systems often include large and complex machines that generate significantly high magnetic fields and create significant constraints on the feasibility of certain surgical interventions. Restrictions can include limited physical access to the patient by a surgeon and/or a surgical robot and/or limitations on the usage of certain electrical and mechanical components in the vicinity of the MRI scanner. Such limitations are inherent in the underlying design of many existing systems and are difficult to overcome.

### SUMMARY

In one general aspect, the present disclosure describes a system including a magnetic resonance imaging (MRI) scanner and a surgical robot. The MRI scanner can include a permanent magnet array. The MRI scanner can define a dome configured to surround a head or extremity of a patient. For example, the MRI scanner can be configured to generate an image of a region of interest of the head of the patient. The MRI scanner can include an opening for accessing the head of the patient. The surgical robot can include robotic arm. The surgical robot can be mounted to the MRI scanner. The surgical robot can be configured to pass through the opening to perform a surgical procedure on the portion of the patient positioned within the dome of the MRI scanner.

### BRIEF DESCRIPTION OF THE DRAWINGS

The various aspects described herein, both as to organization and methods of operation, together with further objects and advantages thereof, may best be understood by reference to the following description, taken in conjunction with the accompanying drawings as follows.

FIG. 1 depicts components of an MRI scanning system including a dome-shaped housing for a magnetic array, the dome-shaped housing surrounding a region of interest therein and further depicting the dome-shaped housing positioned to receive at least a portion of the head of a patient reclined on the table into the region of interest, in accordance with at least one aspect of the present disclosure.

FIG. 1A depicts a patient's head positioned in the region of interest of the MRI scanning system of FIG. 1.

FIG. 2 is a perspective view of an alternative dome-shaped housing for a magnetic array for use with the MRI scanning system of FIG. 1, wherein access apertures are defined in the dome-shaped housing, in accordance with at least one aspect of the present disclosure.

FIG. 3 is a perspective view of an alternative dome-shaped housing for a magnetic array for use with the MRI scanning system of FIG. 1, wherein access apertures and an adjustable gap are defined in the dome-shaped housing, in accordance with at least one aspect of the present disclosure.

FIG. 4 depicts a dome-shaped housing for use with an MRI scanning system having an access aperture in the form of a centrally-defined hole, in accordance with at least one aspect of the present disclosure.

FIG. 5 is a cross-sectional view of the dome-shaped housing of FIG. 4, in accordance with at least one aspect of the present disclosure.

FIG. 6 depicts a control schematic for an MRI system, in accordance with at least one aspect of the present disclosure.

FIG. 7 is a flowchart describing a method for obtaining imaging data from an MRI system, in accordance with at least one aspect of the present disclosure.

FIG. 8 depicts an MRI scanning system and a robotic system, in accordance with at least one aspect of the present disclosure.

FIG. 9 is a perspective view of an MRI-guided surgical robotic system, in accordance with various aspects of the present disclosure.

FIG. 10 is a side view of an MRI-guided surgical robotic system, in accordance with various aspects of the present disclosure.

FIG. 11 is a side view of an MRI-guided surgical robotic system proximate to a patient bed, in accordance with various aspects of the present disclosure.

FIG. 12 is a section view of an MRI-guided surgical robotic system, in accordance with various aspects of the present disclosure.

FIG. 13 is a side view of a portion of an MRI-guided surgical robotic system, in accordance with various aspects of the present disclosure.

FIG. 14 is a perspective view of a fixation device operatively coupled to the head of a patient, in accordance with various aspects of the present disclosure.

FIG. 15 is a perspective view of an MRI-guided surgical robotic system, in accordance with various aspects of the present disclosure.

FIG. 16 is a rear view of an MRI-guided surgical robotic system having a rotatable MRI scanner, in accordance with various aspects of the present disclosure.

FIG. 17 is a front view of a rotatable MRI scanner, in accordance with various aspects of the present disclosure.

FIG. 18 is a rear view of an MRI-guided surgical robotic system having a rotatable MRI scanner, in accordance with various aspects of the present disclosure.

FIG. 19 is a rear view of an MRI-guided surgical robotic system having a rotatable MRI scanner, in accordance with various aspects of the present disclosure.

FIG. 20 is a rear view of a rotatable MRI scanner, in accordance with various aspects of the present disclosure.

FIG. 21 is a side view of an MRI-guided surgical robotic system having a rotatable MRI scanner, in accordance with various aspects of the present disclosure.

FIG. 22 is a side view of an MRI-guided surgical robotic system having a cart-mounted surgical robot, in accordance with various aspects of the present disclosure.

FIG. 23 is a perspective view of an MRI-guided surgical robotic system having a cart-mounted surgical robot, in accordance with various aspects of the present disclosure.

FIG. 24 is a side view of an MRI scanner with guides for positioning a surgical robot, in accordance with various aspects of the present disclosure.

FIG. 25 is a front view of an MRI scanner with guides for positioning a surgical robot, in accordance with various aspects of the present disclosure.

FIG. 26 is a section view of an MRI scanner with guides for positioning a surgical robot, in accordance with various aspects of the present disclosure.

FIG. 27 is a perspective view of a splitable MRI scanner, in accordance with various aspects of the present disclosure.

FIG. 28 is a top view of a splitable MRI scanner, in accordance with various aspects of the present disclosure.

FIG. 29 is a section view of a splittable MRI scanner, in accordance with various aspects of the present disclosure.

FIG. 30 is a section view of a splittable MRI scanner, in accordance with various aspects of the present disclosure.

FIG. 31 is a perspective view of a splittable MRI scanner, in accordance with various aspects of the present disclosure.

FIG. 32 is a section view of a portion of an MRI-guided surgical robotic system having a long-bored MRI scanner, in accordance with various aspects of the present disclosure.

FIG. 33 is a perspective view of an MRI-guided surgical robotic system having a long-bored MRI scanner, in accordance with various aspects of the present disclosure.

FIG. 34 is a perspective view of an MRI scanner having a guide for positioning a surgical robot, in accordance with various aspects of the present disclosure.

FIG. 35 is a perspective view of an MRI scanner having a guide for position a surgical robot, in accordance with various aspects of the present disclosure.

FIG. 36 is a section view of an MRI scanner having a guide for position a surgical robot, in accordance with various aspects of the present disclosure.

FIG. 37 is a flowchart of a method for operating an MRI-guided surgical robotic system, in accordance with various aspects of the present disclosure.

Corresponding reference characters indicate corresponding parts throughout the several views. The exemplifications set out herein illustrate various disclosed embodiments, is one form, and such exemplifications are not to be construed as limiting the scope thereof in any manner.

### DETAILED DESCRIPTION

Applicant of the present application also owns the following patent applications, which are each herein incorporated by reference in their respective entireties:
International Patent Application No. PCT/US2022/72143, titled NEURAL INTERVENTIONAL MAGNETIC RESONANCE IMAGING APPARATUS, filed May 5, 2022 and published on Nov. 10, 2022 as WO2022/236308;
U.S. patent application Ser. No. 18/057,207, titled SYSTEM AND METHOD FOR REMOVING ELECTROMAGNETIC INTERFERENCE FROM LOW-FIELD MAGNETIC RESONANCE IMAGES, filed Nov. 19, 2022;
U.S. patent application Ser. No. 18/147,418, titled MODULARIZED MULTI-PURPOSE MAGNETIC RESONANCE PHANTOM, filed Dec. 28, 2022;
U.S. patent application Ser. No. 18/147,542, titled INTRACRANIAL RADIO FREQUENCY COIL FOR INTRAOPERATIVE MAGNETIC RESONANCE IMAGING, filed Dec. 28, 2022;
U.S. patent application Ser. No. 18/147,556, titled DEEP LEARNING SUPER-RESOLUTION TRAINING FOR ULTRA LOW-FIELD MAGNETIC RESONANCE IMAGING, filed Dec. 28, 2022;
U.S. patent application Ser. No. 18/153,111, titled ACCELERATING MAGNETIC RESONANCE IMAGING USING PARALLEL IMAGING AND ITERATIVE IMAGE RECONSTRUCTION, filed Jan. 11, 2023; and
U.S. patent application Ser. No. 18/153,175, titled FAST T2-WEIGHTED AND DIFFUSION-WEIGHTED CHIRPED-CPMG SEQUENCES, filed Jan. 11, 2023.

Before explaining various aspects of interventional magnetic resonance imaging systems and methods in detail, it should be noted that the illustrative examples are not limited in application or use to the details of construction and arrangement of parts illustrated in the accompanying drawings and description. The illustrative examples may be implemented or incorporated in other aspects, variations and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects and/or examples.

Various aspects are directed to neural interventional magnetic resonance imaging (MRI) devices that allows for the integration of surgical intervention and guidance with an MRI. This includes granting physical access to the area around the patient as well as access to the patient's head with one or more access apertures. In addition, the neural interventional MRI device may allow for the usage of robotic guidance tools and/or traditional surgical implements. In various instances, a neural interventional MRI can be used intraoperatively to obtain scans of a patient's head and/or brain during a surgical intervention, such as a surgical procedure like a biopsy or neural surgery. In other instances, the interventional MRI can be used to image other extremities (e.g. oper or lower limbs) in connection with a robotic surgical procedure.

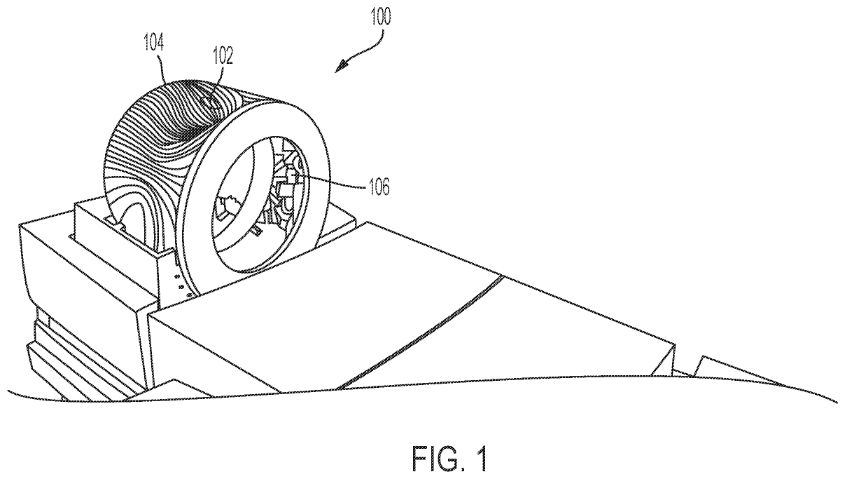

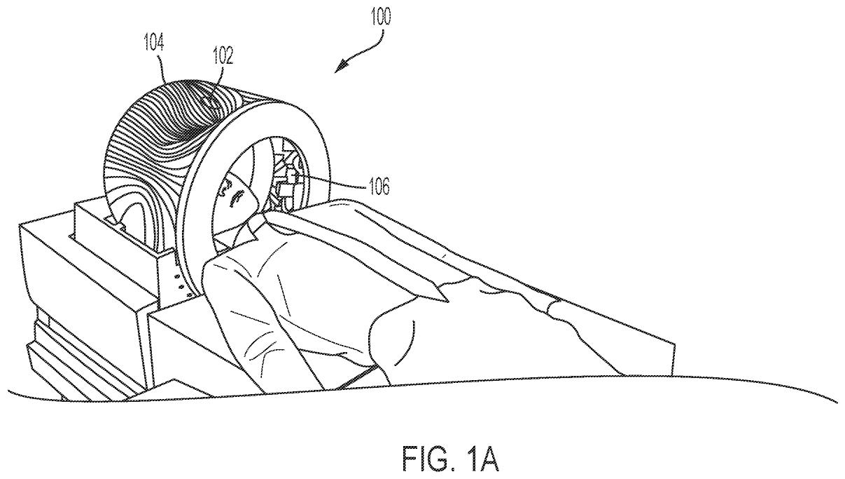

FIG. 1 depicts an MRI scanning system 100 that includes a dome-shaped housing 102 configured to receive a patient's head. The dome-shaped housing 102 can further include at least one access aperture configured to allow access to the patient's head to enable a neural intervention. A space within the dome-shaped housing 102 forms the region of interest for the MRI scanning system 100. Target tissue in the region of interest is subjected to magnetization fields/pulses, as further described herein, to obtain imaging data representative of the target tissue.

For example, referring to FIG. 1A, a patient can be positioned such that his/her head is positioned within the region of interest within the dome-shaped housing 102. The brain can be positioned entirely within the dome-shaped housing 102. In such instances, to facilitate intracranial interventions (e.g. neurosurgery) in concert with MR imaging, the dome-shaped housing 102 can include one or more apertures that provide access to the brain. Apertures can be spaced apart around the perimeter of the dome-shaped housing.

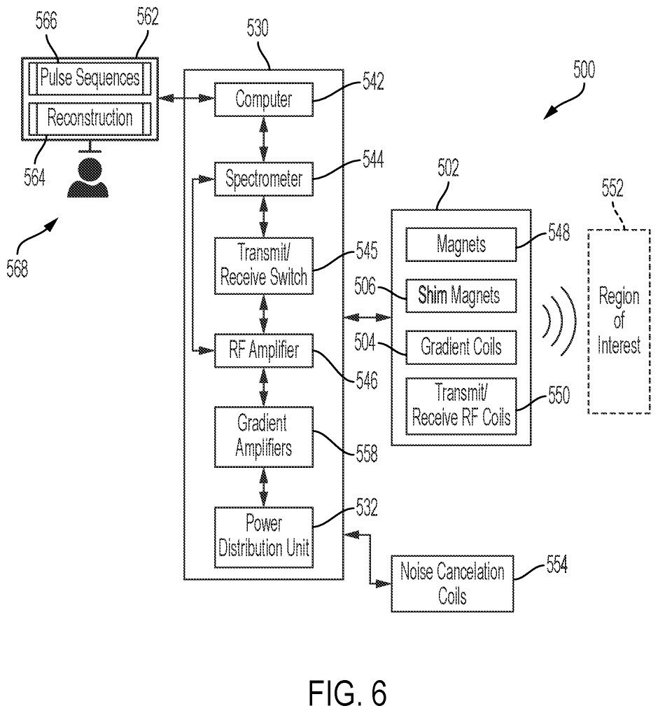

The MRI scanning system 100 can include an auxiliary cart (see, e.g. auxiliary cart 530 in FIG. 6) that houses certain conventional MRI electrical and electronic components, such as a computer, programmable logic controller, power distribution unit, and amplifiers, for example. The MRI scanning system 100 can also include a magnet cart that holds the dome-shaped housing 102, gradient coil(s), and/or a transmission coil, as further described herein. Additionally, the magnet cart can be attached to a receive coil in various instances. Referring primarily to FIG. 1, the dome-shaped housing 102 can further include RF transmission coils, gradient coils 104 (depicted on the exterior thereof), and shim magnets 106 (depicted on the interior thereof). Alternative configurations for the gradient coil(s) 104 and/or shim magnets 106 are also contemplated. In various instances, the shim magnets 106 can be adjustably positioned in a shim tray within the dome-shaped housing 102, which can allow a technician to granularly configure the magnetic flux density of the dome-shaped housing 102.

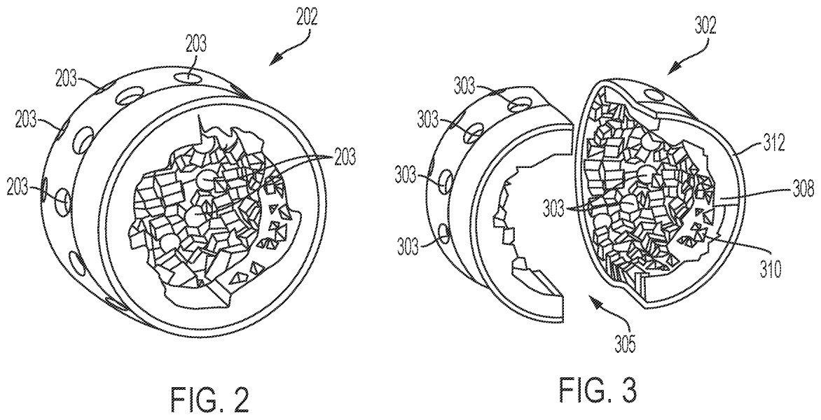

Various structural housings for receiving the patient's head and enabling neural interventions can be utilized with an MRI scanning system, such as the MRI scanning system 100. In one aspect, the MRI scanning system 100 may be outfitted with an alternative housing, such as a dome-shaped housing 202 (FIG. 2) or a two-part housing 302 (FIG. 3) configured to form a dome-shape. The dome-shaped housing 202 defines a plurality of access apertures 203; the two-part housing 302 also defines a plurality of access apertures 303 and further includes an adjustable gap 305 between the two parts of the housing.

In various instances, the housings 202 and 302 can include a bonding agent 308, such as an epoxy resin, for example, that holds a plurality of magnetic elements 310 in fixed positions. The plurality of magnetic elements 310 can be bonded to a structural housing 312, such as a plastic substrate, for example. In various aspects, the bonding agent 308 and structural housing 312 may be non-conductive or diamagnetic materials. Referring primarily to FIG. 3, the two-part housing 302 comprises two structural housings 312. In various aspect, a structural housing for receiving the patient's head can be formed from more than two sub-parts. The access apertures 303 in the structural housing 312 provide a passage directly to the patient's head and are not obstructed by the structural housing 312, bonding agent 308, or magnetic elements 310. The access apertures 303 can be positioned in an open space of the housing 302, for example.

There are many possible configurations of neural interventional MRI devices that can achieve improved access for surgical intervention. Many configurations build upon two main designs, commonly known as the Halbach cylinder and the Halbach dome described in the following article: Cooley et al. (e.g. Cooley, C. Z., Haskell, M. W., Cauley, S. F., Sappo, C., Lapierre, C. D., Ha, C. G., Stockmann, J. P., & Wald, L. L. (2018). Design of sparse Halbach magnet arrays for portable MRI using a genetic algorithm. IEEE transactions on magnetics, 54(1), 5100112. The article “Design of sparse Halbach magnet arrays for portable MRI using a genetic algorithm” by Cooley et al., published in IEEE transactions on magnetics, 54(1), 5100112 in 2018, is incorporated by reference herein in its entirety.

In various instances, a dome-shaped housing for an MRI scanning system, such as the system 100, for example, can include a Halbach dome defining a dome shape and configured based on several factors including main magnetic field B₀ strength, field size, field homogeneity, device size, device weight, and access to the patient for neural intervention. In various aspects, the Halbach dome comprises an exterior radius and interior radius at the base of the dome. The Halbach dome may comprise an elongated cylindrical portion that extends from the base of the dome. In one aspect, the elongated cylindrical portion comprises the same exterior radius and interior radius as the base of the dome and continues from the base of the dome at a predetermined length, at a constant radius. In another aspect, the elongated cylindrical portion comprises a different exterior radius and interior radius than the base of the dome (see e.g. FIGS. 2 and 3). In such instances, the different exterior radius and interior radius of the elongated cylindrical portion can merge with the base radii in a transitional region.

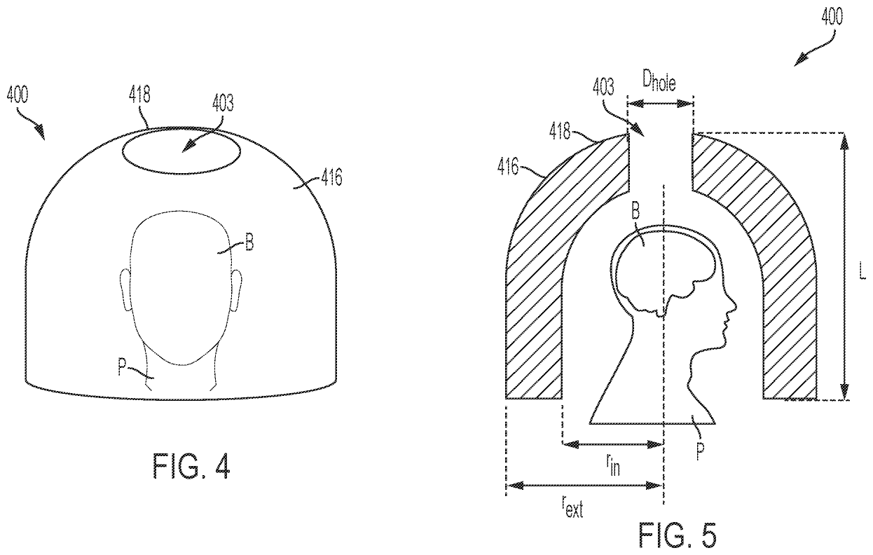

FIG. 4 illustrates an exemplary Halbach dome 400 for an MRI scanning system, such as the system 100, for example, which defines an access aperture in the form of a hole or access aperture 403, where the dome 400 is configured to receive a head and brain B of the patient P within the region of interest therein, and the access aperture 403 is configured to allow access to the patient P to enable neural intervention with a medical instrument and/or robotically-controlled surgical tool, in accordance with at least one aspect of the present disclosure. The Halbach dome 400 can be built with a single access aperture 403 at the top side 418 of the dome 400, which allows for access to the top of the skull while minimizing the impact to the magnetic field. Additionally or alternatively, the dome 300 can be configured with multiple access apertures around the structure 416 of the dome 400, as shown in FIGS. 2 and 3.

The diameter Dₕₒₗₑ of the access aperture 403 may be small (e.g. about 2.54 cm) or very large (substantially the exterior rₑₓₜ diameter of the dome 400). As the access aperture 403 becomes larger, the dome 400 begins to resemble a Halbach cylinder, for example. The access aperture 403 is not limited to being at the apex of the dome 400. The access aperture 403 can be placed anywhere on the surface or structure 416 of the dome 400. In various instances, the entire dome 400 can be rotated so that the access aperture 403 can be co-located with a desired physical location on the patient P.

FIG. 5 depicts relative dimensions of the Halbach dome 400, including a diameter Dₕₒₗₑ of the access aperture 403, a length L of the dome 400, and an exterior radius rₑₓₜ and an interior radius rᵢₙ of the dome 400. The Halbach dome 400 comprises a plurality of magnetic elements that are configured in a Halbach array and make up a magnetic assembly. The plurality of magnetic elements may be enclosed by the exterior radius rₑₓₜ and interior radius rᵢₙ in the structure 416 or housing thereof. In one aspect, example dimensions may be defined as: rᵢₙ=19.3 cm; rₑₓₜ=23.6 cm; L=38.7 cm; and 2.54 cm≤D<19.3 cm.

Based on the above example dimensions, a Halbach dome 400 with an access aperture 403 may be configured with a magnetic flux density B₀ of around 72 mT, and an overall mass of around 35 kg. It will be appreciated that the dimensions may be selected based on particular applications to achieve a desired magnetic flux density B₀, total weight of the Halbach dome 400 and/or magnet cart, and geometry of the neural intervention access aperture 403.

In various aspects, the Halbach dome 400 may be configured to define multiple access apertures 403 placed around the structure 416 of the dome 400. These multiple access apertures 403 may be configured to allow for access to the patient's head and brain B using tools (e.g., surgical tools) and/or a surgical robot.

In various aspects, the access aperture 403 may be adjustable. The adjustable configuration may provide the ability for the access aperture 403 to be adjusted using either a motor, mechanical assist, or a hand powered system with a mechanical iris configuration, for example, to adjust the diameter Dₕₒₗₑ of the access aperture 403. This would allow for configuration of the dome without an access aperture 403, conducting an imaging scan, and then adjusting the configuration of the dome 400 and mechanical iris thereof to include the access aperture 403 and, thus, to enable a surgical intervention therethrough.

Halbach domes and magnetic arrays thereof for facilitating neural interventions are further described in International Patent Application No. PCT/US2022/72143, titled NEURAL INTERVENTIONAL MAGNETIC RESONANCE IMAGING APPARATUS, filed May 5, 2022, which is incorporated by reference herein in its entirety.

Referring now to FIG. 6, a schematic for an MRI system 500 is shown. The MRI scanning system 100 (FIG. 1) and the various dome-shaped housings and magnetic arrays therefor, which are further described herein, for example, can be incorporated into the MRI system 500, for example. The MRI system 500 includes a housing 502, which can be similar in many aspects to the dome-shaped housings 102 (FIG. 1), 202 (FIG. 2), and/or 302 (FIG. 3), for example. The housing 502 is dome-shaped and configured to form a region of interest, or field of view, 552 therein. For example, the housing 502 can be configured to receive a patient's head in various aspects of the present disclosure.

The housing 502 includes a magnet assembly 548 having a plurality of magnets arranged therein (e.g. a Halbach array of magnets). In various aspect, the main magnetic field B₀, generated by the magnetic assembly 548, extends into the field of view 552, which contains an object (e.g. the head of a patient) that is being imaged by the MRI system 500.

The MRI system 500 also includes RF transmit/receive coils 550. The RF transmit/receive coils 550 are combined into integrated transmission-reception (Tx/Rx) coils. In other instances, the RF transmission coil can be separate from the RF reception coil. For example, the RF transmission coil(s) can be incorporated into the housing 502 and the RF reception coil(s) can be positioned within the housing 502 to obtain imaging data.

The housing 502 also includes one or more gradient coils 504, which are configured to generate gradient fields to facilitate imaging of the object in the field of view 552 generated by the magnet assembly 548, e.g., enclosed by the dome-shaped housing and dome-shaped array of magnetic elements therein. Shim trays adapted to receive shim magnets 506 can also be incorporated into the housing 502.

During the imaging process, the main magnetic field B₀ extends into the field of view 552. The direction of the effective magnetic field (B₁) changes in response to the RF pulses and associated electromagnetic fields transmitted by the RF transmit/receive coils 550. For example, the RF transmit/receive coils 550 may be configured to selectively transmit RF signals or pulses to an object in the field of view 552, e.g. tissue of a patient's brain. These RF pulses may alter the effective magnetic field experienced by the spins in the sample tissue.

The housing 502 is in signal communication with an auxiliary cart 530, which is configured to provide power to the housing 502 and send/receive control signals to/from the housing 502. The auxiliary cart 530 includes a power distribution unit 532, a computer 542, a spectrometer 544, a transmit/receive switch 545, an RF amplifier 546, and gradient amplifiers 558. In various instances, the housing 502 can be in signal communication with multiple auxiliary carts and each cart can support one or more of the power distribution unit 532, the computer 542, the spectrometer 544, the transmit/receive switch 545, the RF amplifier 546, and/or the gradient amplifiers 558.

The computer 542 is in signal communication with a spectrometer 544 and is configured to send and receive signals between the computer 542 and the spectrometer 544. When the object in the field of view 552 is excited with RF pulses from the RF transmit/receive coils 550, the precession of the object results in an induced electric current, or MR current, which is detected by the RF transmit/receive coils 550 and sent to the RF amplifier 546. The RF amplifier 546 is configured to boost or amplify the excitation data signals and send them to the spectrometer 544. The spectrometer 544 is configured to send the excitation data to the computer 542 for storage, analysis, and image construction. The computer 542 is configured to combine multiple stored excitation data signals to create an image, for example. In various instances, the computer 542 is in signal communication with at least one database 562 that stores reconstruction algorithms 564 and/or pulse sequences 566. The computer 542 is configured to utilize the reconstruction algorithms to generate an MR image 568.

From the spectrometer 544, signals can also be relayed to the RF transmit/receive coils 550 in the housing 502 via an RF power amplifier 546 and the transmit/receive switch 545 positioned between the spectrometer 544 and the RF power amplifier 546. From the spectrometer 544, signals can also be relayed to the gradient coils 504 in the housing 502 via a gradient power amplifier 558. For example, the RF power amplifier 546 is configured to amplify the signal and send it to RF transmission coils 550, and the gradient power amplifier 558 is configured to amplify the gradient coil signal and send it to the gradient coils 504.

In various instances, the MRI system 500 can include noise cancellation coils 554. For example, the auxiliary cart 530 and/or computer 542 can be in signal communication with noise cancellation coils 554. In other instances, the noise cancellation coils 554 can be optional. For example, certain MRI systems disclosed herein may not include supplemental/auxiliary RF coils for detecting and canceling electromagnetic interference, i.e. noise.

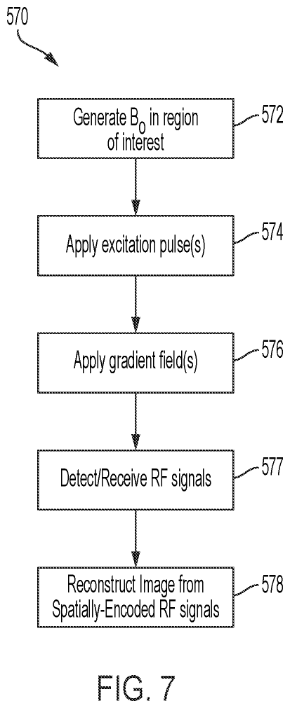

A flowchart depicting a process 570 for obtaining an MRI image is shown in FIG. 7. The flowchart can be implemented by the MRI system 500, for example. In various instances, at block 572, the target subject (e.g. a portion of a patient's anatomy), is positioned in a main magnetic field B₀ in an interest of region (e.g. region of interest 552), such as within the dome-shaped housing of the various MRI scanners further described herein (e.g. magnet assembly 548). The main magnetic field B₀ is configured to magnetically polarize the hydrogen protons (1H-protons) of the target subject (e.g. all organs and tissues) and is known as the net longitudinal magnetization M₀. It is proportional to the proton density (PD) of the tissue and develops exponentially in time with a time constant known as the longitudinal relaxation time T1 of the tissue. T1 values of individual tissues depend on a number of factors including their microscopic structure, on the water and/or lipid content, and the strength of the polarizing magnetic field, for example. For these reasons, the T1 value of a given tissue sample is dependent on age and state of health.

At block 574, a time varying oscillatory magnetic field B₁, i.e. an excitation pulse, is applied to the magnetically polarized target subject with a RF coil (e.g. RF transmit/receive coil 550). The carrier frequency of the pulsed B₁ field is set to the resonance frequency of the 1H-proton, which causes the longitudinal magnetization to flip away from its equilibrium longitudinal direction resulting in a rotated magnetization vector, which in general can have transverse as well as longitudinal magnetization components, depending on the flip angle used. Common B₁ pulses include an inversion pulse, or a 180-degree pulse, and a 90-degree pulse. A 180-degree pulse reverses the direction of the 1H-proton's magnetization in the longitudinal axis. A 90-degree pulse rotates the 1H-proton's magnetization by 90 degrees so that the magnetization is in the transverse plane. The MR signals are proportional to the transverse components of the magnetization and are time varying electrical currents that are detected with suitable RF coils. These MR signals decay exponentially in time with a time constant known as the transverse relaxation time T2, which is also dependent on the microscopic tissue structure, water/lipid content, and the strength of the magnetic field used, for example.

At block 576, the MR signals are spatially encoded by exposing the target subject to additional magnetic fields generated by gradient coils (e.g. gradient coils 504), which are known as the gradient fields. The gradient fields, which vary linearly in space, are applied for short periods of time in pulsed form and with spatial variations in each direction. The net result is the generation of a plurality of spatially encoded MR signals, which are detected at block 577, and which can be reconstructed to form MR images depicting slices of the examination subject. A RF reception coil (e.g. RF transmit/receive coil 550) can be configured to detect the spatially-encoded RF signals. Slices may be oriented in the transverse, sagittal, coronal, or any oblique plane.

At block 578, the spatially encoded signals of each slice of the scanned region are digitized and spatially decoded mathematically with a computer reconstruction program (e.g. by computer 542) in order to generate images depicting the internal anatomy of the examination subject. In various instances, the reconstruction program can utilize an (inverse) Fourier transform to back-transforms the spatially-encoded data (k-space data) into geometrically decoded data.

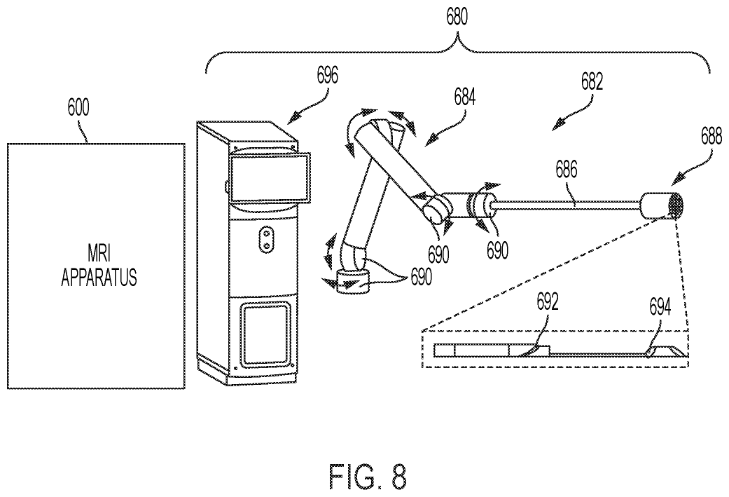

FIG. 8 depicts a graphical illustration of a robotic system 680 that may be used for neural intervention with an MRI scanning system 600. The robotic system 680 includes a computer system 696 and a surgical robot 682. The MRI scanning system 600 can be similar to the MRI system 500 and can include the dome-shaped housing and magnetic arrays having access apertures, as further described herein. For example, the MRI system 500 can include one or more access apertures defined in a Halbach array of magnets in the permanent magnet assembly to provide access to one or more anatomical parts of a patient being imaged during a medical procedure. In various instances, a robotic arm and/or tool of the surgical robot 682 is configured to extend through an access aperture in the permanent magnet assembly to reach a patient or target site. Each access aperture can provide access to the patient and/or surgical site. For example, in instances of multiple access apertures, the multiple access apertures can allow access from different directions and/or proximal locations.

In accordance with various embodiments, the robotic system 680 is configured to be placed outside the MRI system 600. As shown in FIG. 8, the robotic system 680 can include a robotic arm 684 that is configured for movements with one or more degrees of freedom. In accordance with various embodiments, the robotic arm 684 includes one or more mechanical arm portions, including a hollow shaft 686 and an end effector 688. The hollow shaft 686 and end effector 688 are configured to be moved, rotated, and/or swiveled through various ranges of motion via one or more motion controllers 690. The double-headed curved arrows in FIG. 8 signify exemplary rotational motions produced by the motion controllers 690 at the various joints in the robotic arm 684.

In accordance with various embodiments, the robotic arm 684 of the robotic system 682 is configured for accessing various anatomical parts of interest through or around the MRI scanning system 600. In accordance with various embodiments, the access aperture is designed to account for the size of the robotic arm 684. For example, the access aperture defines a circumference that is configured to accommodate the robotic arm 684, the hollow shaft 686, and the end effector 688 therethrough. In various instances, the robotic arm 684 is configured for accessing various anatomical parts of the patient from around a side of the magnetic imaging apparatus 600. The hollow shaft 686 and/or end effector 688 can be adapted to receive a robotic tool 692, such as a biopsy needle having a cutting edge 694 for collecting a biopsy sample from a patient, for example.

The reader will appreciate that the robotic system 682 can be used in combination with various dome-shaped and/or cylindrical magnetic housings further described herein. Moreover, the robotic system 682 and robotic tool 692 in FIG. 8 are exemplary. Alternative robotic systems can be utilized in connection with the various MRI systems disclosed herein. Moreover, handheld surgical instruments and/or additional imaging devices (e.g. an endoscope) and/or systems can also be utilized in connection with the various MRI systems disclosed herein.

In various aspects of the present disclosure, the MRI systems described herein can comprise low field MRI (LF-MRI) systems. In such instances, the main magnetic field B₀ generated by the permanent magnet assembly can be less than 1.0 T, such as, between 0.1 T and 1.0 T, for example. In other instances, the MRI systems described herein can comprise ultra-low field MRI (ULF-MRI) systems. In such instances, the main magnetic field B₀ generated by the permanent magnet assembly can be between 0.03 T and 0.1 T, for example.

Higher magnetic fields, such as magnetic fields above 1.0 T, for example, can preclude the use of certain electrical and mechanical components in the vicinity of the MRI scanner. For example, the existence of surgical instruments and/or surgical robot components comprising metal, specially ferrous metals, can be dangerous in the vicinity of higher magnetic fields because such tools can be drawn toward the source of magnetization. Moreover, higher magnetic fields often require specifically-designed rooms with additional precautions and shielding to limit magnetic interference. Despite the limitations on high field MRI systems, low field and ultra-low field MRI systems present various challenges to the acquisition of high quality images with sufficient resolution for achieving the desired imaging objectives.

LF- and ULF-MRI systems generally define an overall magnetic field homogeneity that is relatively poor in comparison to higher field MRI systems. For example, a dome-shaped housing for an array of magnets, as further described herein, can comprise a Halbach array of permanent magnets, which generate a magnetic field B₀ having a homogeneity between 1,000 ppm and 10,000 ppm in the region of interest in various aspects of the present disclosure.

### MRI-Guided Surgical Robotic Devices, Systems, and Methods

Having described aspects of various MRI and robotic devices, systems, and methods, such as the MRI scanning system 100 (FIG. 1), the housing 202 (FIG. 2), the housing 302 (FIG. 3), the Halbach dome 400 (FIG. 4), the MRI system 500 (FIG. 6), the process 570 (FIG. 7), and the robotic system 680 (FIG. 8), the disclosure turns to describe various co-operative MRI systems and robotic platforms (e.g. robotic devices, systems, and methods). Any aspects of the various MRI and robotic devices, systems, and methods described above may be included in or otherwise applied with the various co-operative MRI systems and robotic platforms described below. Likewise, any aspects of the MRI and robotic devices, systems, and methods described below may be included in or otherwise applied to the above-described robotic platforms and MRI systems.

Conventional MRI scanners are typically applied as a tool for noninvasive diagnosis and intervention. For example, MRI scanners have been applied for imaging highly-sensitive organs and tissue such as the brain. However, the infrastructure requirements of conventional MRI scanners can generally make them costly and immobile, thereby limiting their deployment and accessibility. For example, conventional MRI scanners often use cryogenically cooled electromagnets having a field strength of 1 T to 3 T. Furthermore, conventional MRI scanners often require specialized siting requirements for power, cooling, and magnetic and RF shielding. Thus, conventional MRI scanners are typically sited permanently, requiring multiple dedicated rooms, and are typically expensive. Moreover, the field strength of 1 T to 3 T that is often produced by conventional MRI scanners can cause complication for some surgical applications.

Many clinical applications may therefore benefit from an MRI scanner that is portable, low cost, and/or low field. Further, Minimally Invasive Surgery (MIS) may benefit from intra-operative MRI imaging and surgical guidance, for example, using a portable, low-cost, and/or low-field MRI scanner. Yet further, MIS may benefit from combining a surgical robot with a portable, low-cost, and/or low-field MRI scanner.

However, portable, low-cost, and/or low-field MRI scanners can have limitations related to field strength, homogeneity, and/or small-bore diameter, which can greatly reduce imaging quality and limit clinical applications. For example, some existing low-field MRI scanners use a permanent magnetic array (PMA) to reduce power and siting requirements, lower cost, and increase portability. PMA designs may use H-shaped or C-shaped magnets. Typical PMA designs often lack the magnet strength to create an imageable field of 55-70 mT when combined with an MRI scanner having a bore diameter large enough for surgical applications. For example, typical PMA designs can require the magnet to be tighter to the patient to create the desired strength, which constrains the bore diameter thereby disallowing or making difficult the use of surgical modalities requiring fixation devices, intubation, and drapes such as cranial fixation.

Thus, many of the existing conventional MRI scanners and low-field MRI devices have limited or no use related to surgical or intra-operative guidance applications such as during a robotic surgical procedure, for example. Accordingly, there exists a need for a portable, low-cost, and/or low-field MRI scanner that can be used in surgical or intra-operative guidance applications. Moreover, there exists a need for a portable, low-cost, and/or low-field MRI scanner that can be used in combination with a surgical robot.

According to various aspects, the present disclosure provides MRI-guided surgical robotic devices, system, and related methods. In some aspects, an MRI-guided surgical robotic system can include an MRI scanner. The MRI scanner can include a magnet array (e.g., a B₀ magnet array). The magnet array can be dome-shaped (e.g., a Halbach array, a modified Halbach array, etc.). The magnet array can be optimized to a shape of a patient's head (e.g. a head-optimized Halbach dome) and/or a patient's extremity while maximizing the resulting field strength. The magnet array can generate a magnetic field with a high flux density in a region of interest (ROI).

In one aspect, the magnet array can include a modified Halbach array. The modified Halbach array can be configured to generate a magnetic field B₀ that is aligned axially to a bore axis of the array. (e.g., patient head-to-toe direction in neurosurgery).

The magnet array can have a bore dimeter. The optimization of the magnet array and the resulting high field strength can enable the bore diameter to be wide enough for compatibility with standard clinical modalities often required for surgical interventions, such as cranial fixation and surgical tables.

In one aspect, the MRI-guided surgical robotic system can include a surgical robot. The surgical robot can comprise an end effector. The surgical robot can be mounted or otherwise attached to the MRI scanner.

In one aspect, the MRI scanner can have an opening or openings for surgical access to the patient's head and/or the patient's extremity by a clinician and/or surgical robot. In one aspect, the opening can be a slot.

In one aspect, the MRI scanner can be configured to rotate (e.g., roll) about the bore axis of the magnet array. The bore axis can be aligned with the head-to-toe axis of the patient's body. For example, the domed housing of the MRI scanner is structured to roll about a longitudinal axis aligned with the patient's body in various aspects of the present disclosure. The rotation of the MRI scanner can be enabled by the dome shape of the magnet array. For example, the rim of the dome can comprise a geared ring or annular rail for rotatably supporting the dome relative to the wheeled cart of the MRI system. The rotation of the MRI scanner can cause the slot and/or other opening(s) on the MRI scanner to be positioned to a desired longitudinal coordinate (e.g., a longitudinal coordinate of the patient aligned with designated lesions, entry points, or planned targets). Positioning the slot and/or other opening(s) by rotation of the MRI scanner can enable surgical-related access.

For example, an end-effector of a surgical robot and/or other surgical tools can pass through the slot and/or other opening(s) to an MRI region of interest (ROI). As another example, an end-effector of a surgical robot and/or other surgical tools can pass through the slot and/or other opening(s) to access a target limb or tissue (e.g., entry points or fiducial markers on the skull) during registration, verification, and surgical operation. As another example, the slot and/or other opening(s) can provide visual access to a surgeon. As yet another example, in aspects where the MRI scanner comprises a slot, the slot design can allow access to any coordinate on a hemisphere of the patient by coordinating the rotation of the MRI scanner with a longitudinal coordinate. The slot and/or other opening(s) in the MRI scanner can be configured to allow access to, for example, a cranium of a patient by the surgical robot.

In one aspect, the surgical robot can include and/or be attached to an adjustable arm (e.g., anthropomorphic or parallel).

In one aspect, the MRI scanner can include an arc sliding guide for positioning the surgical robot.

In one aspect, the arm (e.g., adjustable arm) can be mounted onto and/or rotate with the MRI frame. In another aspect, the arm can be installed on the cart (e.g., independent of the MRI orientation.)

In one aspect, the MRI scanner and/or the magnet array can define a non-spherical dome. For example, the MRI scanner and/or the magnet array can define a radially elongated dome (e.g., shorter (top to bottom) and wider (left to right) than conventional dome shapes). As another example, the radius of the opening of the dome can be greater than the radius along the bore axis of the dome.

In one aspect, the MRI-guided surgical robotic system MRI can be configured to generate MRI images for guiding the surgical robot in real-time. In another aspect, the MRI-guided surgical robotic system MRI can be configured to provide diagnostic imaging.

In one aspect, the MRI-guided surgical robotic system can include a portable cart. The MRI scanner and/or the surgical robot can be mounted on or otherwise positioned on the portable cart. The cart can include integrated electronics and/or control modules for operating and/or controlling the MRI scanner and/or the surgical robot. The portable cart can be compact and light enough to meet clinical sizing standards and/or requirements. The portable cart can be sized to be brought to the patient (e.g., positioned proximate to a patient platform, a patient bed) in an emergency room, surgical theatre, or other clinical setting. The portable cart can include a set of passive or active quick setup/release wheels and brakes to enable the system to be moved onto or away from the patient's bed within a very short time. This can provide the benefit of allowing a surgeon easy access in any kind of emergency situation, since the portable cart can be quickly and easily moved away from the patient. In some aspects, the wheels are omni directional allowing easier movement and guiding of the portable cart.

In one aspect, the MRI-guided surgical robotic system does not require special power, cooling, or a permanent siting infrastructure.

The devices, systems, and methods provided herein can provide various benefits. For example, the devices, systems, and methods provided herein can be used to produce acceptable MR imaging quality for intra-operative guidance of a single or multi-port robot used in robot-assisted surgery.

As another example, the devices, systems, and methods provided herein can offer a portable MRI modality that may not require special shielding or siting infrastructure for power or cooling.

As yet another example, the devices, systems, and methods provided herein can utilize a dome-shaped scanner having interior dimensions optimized for surgical interventions, such a surgical interventions using fixation devices, intubation, drapes, and/or patient positioning within the scanner.

As yet another example, the devices, systems, and methods provided herein can be compatible with existing surgical tables.

As yet another example, the device systems, and methods provided here can enable surgical-related access to a patient's limb extremities, such as cranial hemisphere, hand, or foot, through openings in the MRI structure with minimal reduction in imaging quality. In at least one aspect, the openings subtract from the MRI main magnet PMA.

As yet another example, the devices, systems, and methods provided herein can enable full integration of an image guided intra-operative MR compatible surgical robot.

As yet another example, the devices, systems, and methods provided herein can enable surgical-related access to a desired coordinate in the patient limb extremities by rotating the MRI and pre-adjusting coarse and fine movements of the robot.

As yet another example, the devices, systems, and methods provided herein can enable an improved (e.g., faster) surgical fast workflow and improved surgical outcomes compared to existing devices, systems, and methods.

As yet another example, dimensions of the MRI scanner and/or magnet array, such as a relatively large bore diameter and large patient opening with a short depth can enable the MRI scanner to be used with fixation devices and/or existing surgical tables.

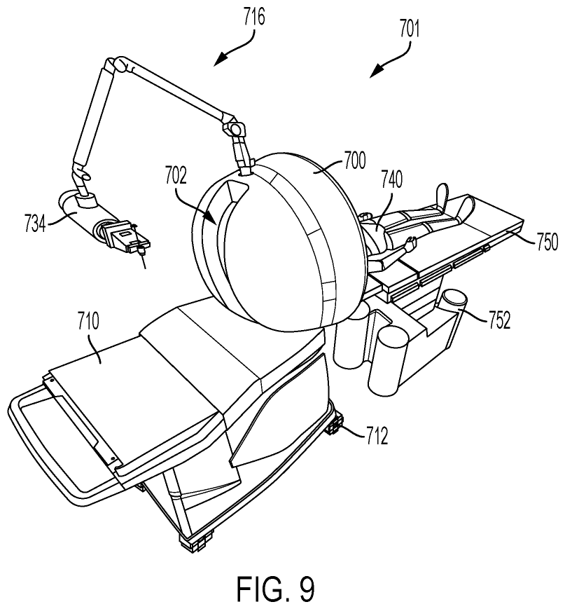

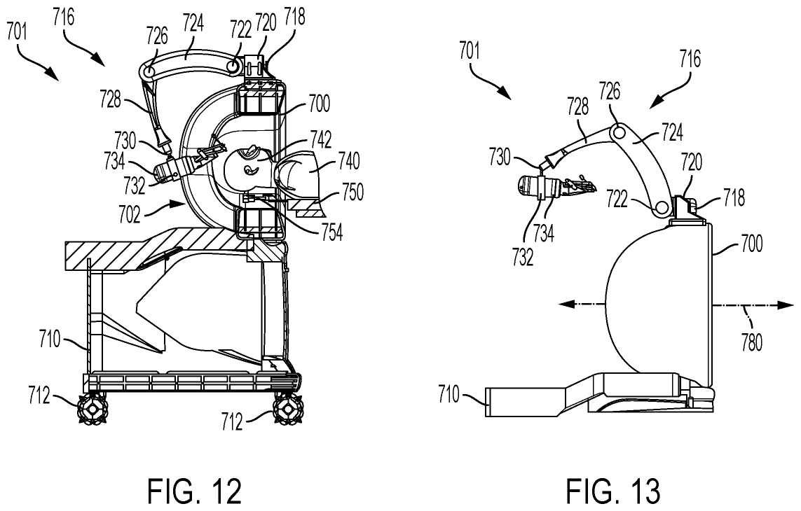

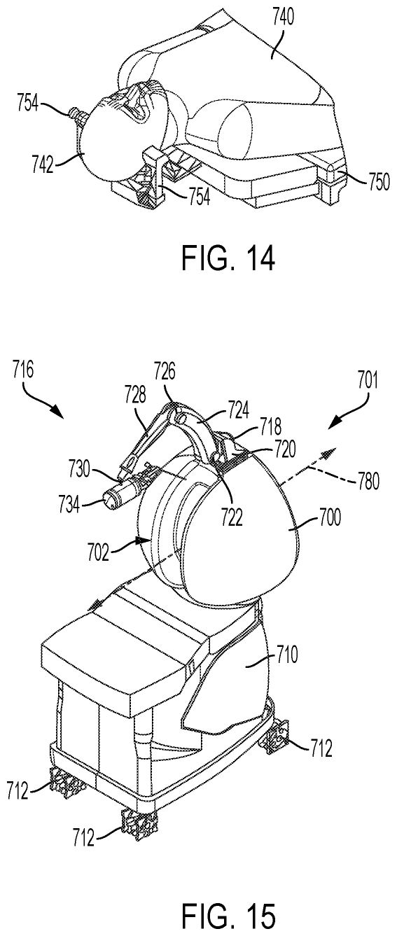

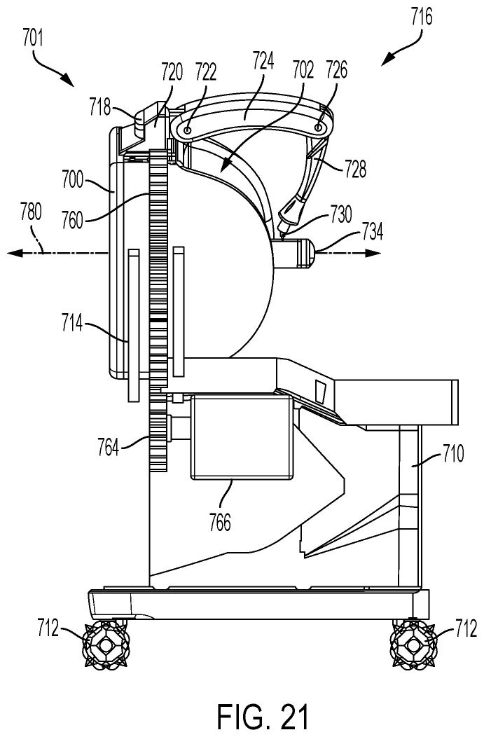

FIGS. 9-21 illustrate an MRI-guided surgical robotic system 701, according to at least one aspect of the present disclosure. The MRI-guided surgical robotic system 701 is sometimes referred to herein at the system 701. Referring primarily to FIG. 9, the system 701 includes a portable low-field MRI scanner 700 (e.g. MRI scanner 100 comprising Halbach dome 400), an image-guided MR-compatible robot 734 (the surgical robot 734) and a cart 710. The surgical robot 734 is positionable by an adjustable arm 716 that is mounted on (or otherwise attached to) the MRI scanner 700, which is in turn supported by the cart 710. The surgical robot 734 is an MR-compatible robot. In at least one aspect, the arm 716 has at least two positional degrees of freedom (DOFs) and at least two DOFs of force feedback. The at least two DOFs allow the arm 716 to reach and position the surgical robot 734 though an opening in the dome-shaped housing of the MRI scanner 700 as described in more detail in regard to FIGS. 12-14. For example, the arm 716 and surgical robot 734 can be positioned in and out of an opening in the dome-shaped housing of the MRI scanner 700. The domed housing of the MRI scanner 700 comprises a wall forming the dome and housing the array of permanent magnets therein (as well as additional components of the MRI scanner/scanning system) The domed geometry is further described herein. The wall extends to a rim forming a boundary of the dome. A portion of a patient's anatomy can extend through the open end of the dome, i.e. past the rim. For example, a patient's upper torso and/or neck can extend past the rim and into the imaging region defined within the domed housing. At least a portion of the wall comprises a curved wall forming the closed end of the dome opposite the rim/open end of the dome. The rim and rimmed opening is separate from the access opening or aperture defined through the housing. The access opening can be defined through the wall and, in various instances, through the solid curved wall of the domed housing. Various access aperture geometries and configurations are further described herein.

FIG. 9 depicts the system 701 positioned proximate to a patient table 750. The patient table 750 is shown supporting a patient 740. In at least one aspect, the cart 710 can be releasably attached (e.g. attached and detached) from the patient table 750.

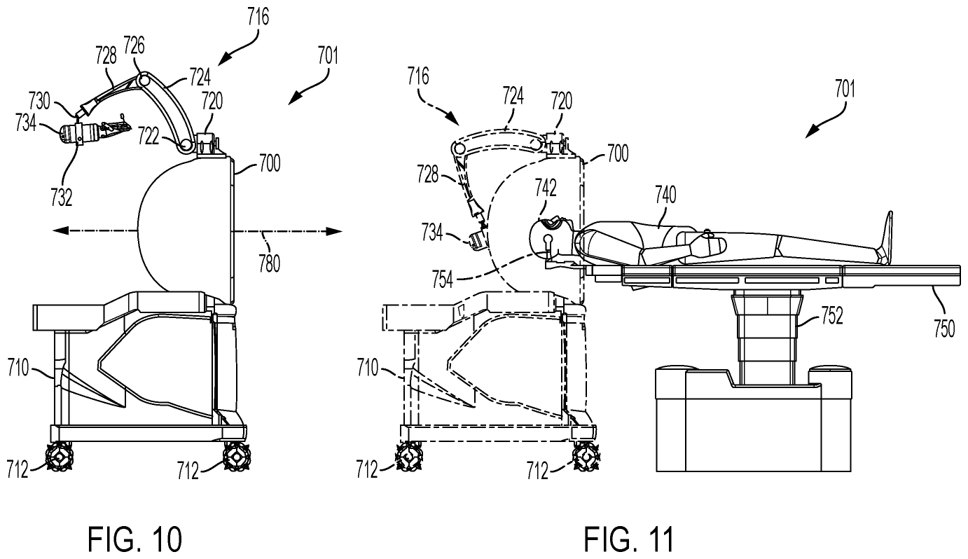

Referring primarily to FIGS. 10 and 11, the patient table 750 can include an extendable column 752 (e.g., a telescoping column) that is configured to adjust the height of patient table 750. Adjusting the height of the patient table 750 can adjust a position of a head 742 of the patient 740 relative to the MRI scanner 700. The cart 710 carrying the MRI scanner 700, the arm 716, and the surgical robot 734 can be positioned relative to (e.g. proximate to) the patient table 750 such that the MRI scanner 700 surrounds the head 742 of the patient 740 such that the patient's skull is located within the region of interest of the MRI scanner 700. The positioning of the MRI scanner 700 relative to the patent 740, as depicted by FIG. 11, can enable efficient use of the magnetic flux generated by the MRI scanner 700, thereby positioning the MRI scanner 700 for optimum imaging of the head 742 of the patient 740. The cart 710 can include quick setup/release wheels 712 operably coupled to the cart 710. In at least one aspect, the wheels 712 can be locked to hold the MRI scanner 700 in position relative to the patient table 750. In an additional or alternative aspect, the cart 710 can attach to the patient table 750. The wheels 712 can provide flexibility for quickly moving the cart toward or away from the patient's table 750. In some aspects, the wheels 712 are omni directional. In an alternative aspect, the wheels 712 are configured to only move in one direction. In various instances, as the cart 710 is moved toward the table 750, the MRI scanner 700 and robot 734 are moved as a unit toward the table 750 and patient positioned thereon.

FIG. 11 illustrates the MRI scanner 700 being used with a patient table 750. Patient table 750 is one example of a patient table that can be used with the MRI scanner 700. There are a plurality of patient tables that can be used with the MRI scanner 700 and patient table 750 is one non-limiting example.

Referring primarily to FIGS. 12-14, the MRI scanner 700 can include an opening 702. The opening 702 can include any suitable shape, such as, for example, a circular hole or a substantially rectangular slot. By controlling the arm 716 (e.g., by actuating the joints/DOFs and positioning a first portion 724 and a second portion 728 of the arm 716), the surgical robot 734 is movable inside the opening 702. The opening 702 can include a space suitable for pre-adjustment of the surgical robot 734 and/or for the surgical staff to perform manual procedures. The arm 716 can be configured to provide coarse yet smooth movements of the surgical robot 734. For example, the surgical robot 734 can be secured to the arm 716 by passive and/or active joints on the arm 716, including but not limited to a shoulder joint 722, an elbow joint 726, and/or a wrist joint 730. Each joint of the arm 716 can include a motor that can be controlled to rotate the joint and move arm 716 to position the surgical robot 734.

The arm 716 can include a base stand 720 that can include fasteners or actuators of any type. For example, the base stand 720 can include a base joint 718 for locking or otherwise coupling the shoulder joint 722 to the base stand 720. A collar 732 can fix or otherwise couple the robot 734 to the arm 716.

In some aspects, the base stand 720 is fixedly attached to the housing of the MRI scanner 700. In some alternative aspects, the base stand 720 can move along a guide that is attached to the housing of the MRI scanner 700, as described in regard to FIGS. 25 and 26. The guide can allow the base stand 720 to move relative to the housing of the MRI scanner 700. By moving along the guide, the entire arm 716 and surgical robot 734 can move relative to the housing of the MRI scanner 700.

In some aspects, an MR-compatible head fixation device 754 can be included on the patient table 750 for holding the head 742 in place during the operation. As such, the opening in the MRI scanner 700 is sized to allow the head fixation device 754 to fit inside of the MRI scanner 700.

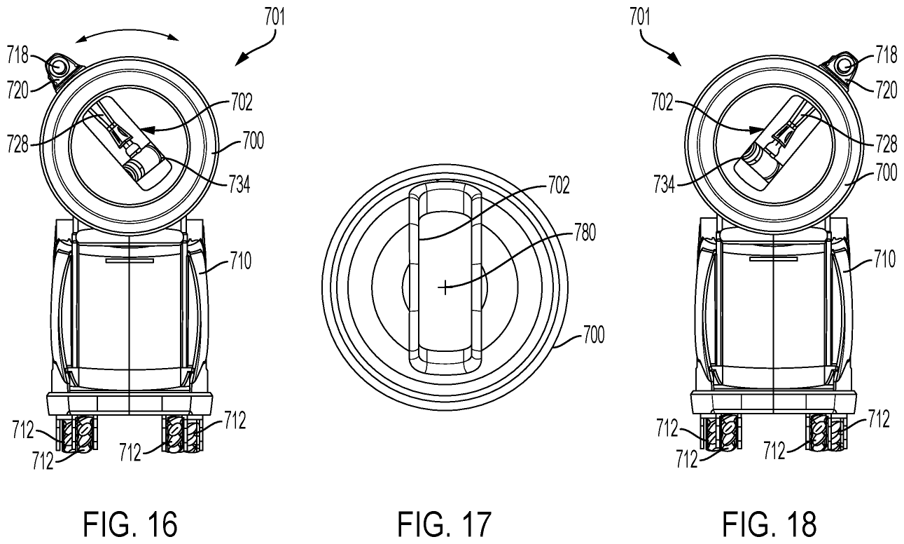

Referring primarily to FIGS. 15-18, the opening 702 is configured as a slot. The MRI scanner 700 can be rotated about an axis 780 (e.g., a bore axis, a longitudinal axis) of the MRI scanner 700 to adjust the orientation of the opening 702. In at least one aspect, the axis 780 goes through the center of the MRI scanner 700 as shown in FIG. 17. For example, FIG. 15 depicts the MRI scanner 700 positioned such that the opening 702 is vertically oriented (e.g. rotated 0°). Transitioning to FIG. 16, the MRI scanner 700 can be repositioned (rotated −45°) such that the opening 702 is oriented at an angle compared to the configuration depicted by FIG. 15. In some aspects, the MRI scanner can be rotated to any angle to achieve a desired orientation of the opening 702 (e.g., 45° as depicted by FIG. 18, 90°, 180°). The opening 702 can be configured as a slot having a width and/or a minimum width that is larger than an outer diameter and/or a maximum diameter of the surgical robot 734. Owing the size and orientation of the slot 702, rotation of the domed housing can provide unrestrained access to locations within the domed housing for surgical intervention.

Referring primarily to FIGS. 16-21, the opening is configured as a slot. The MRI scanner 700 can be rotated about an axis 780 (e.g., a bore axis, a longitudinal axis, see FIG. 17) of the MRI scanner 700 to adjust the orientation of the opening 702 and the orientation of the arm 716 and/or the surgical robot 734. Adjusting the orientation of the opening 702 can allow surgical-related access to various portions of the head 742 of the patient 740. For example, the MRI scanner 700 can be rotated to orient the opening 702 (e.g., the slot) and the surgical robot 734 to access a designated lesion, entry point, and/or planned target coordinate on the head 742 of the patient 740.

In at least one aspect, the surgical robot 734 and the MRI scanner 700 are cooperatively utilized to perform a surgical procedure. For example, the MRI scanner 700 can be used to generate an image in real-time that guides the surgical robot 734 during a surgical procedure. In at least one aspect, the surgical robot 734 is within the MRI scanner 700 during the data collection to generate an image. In an alternative aspect, the surgical robot 734 is removed from within the MRI scanner 700 during the data collection to generate an image.

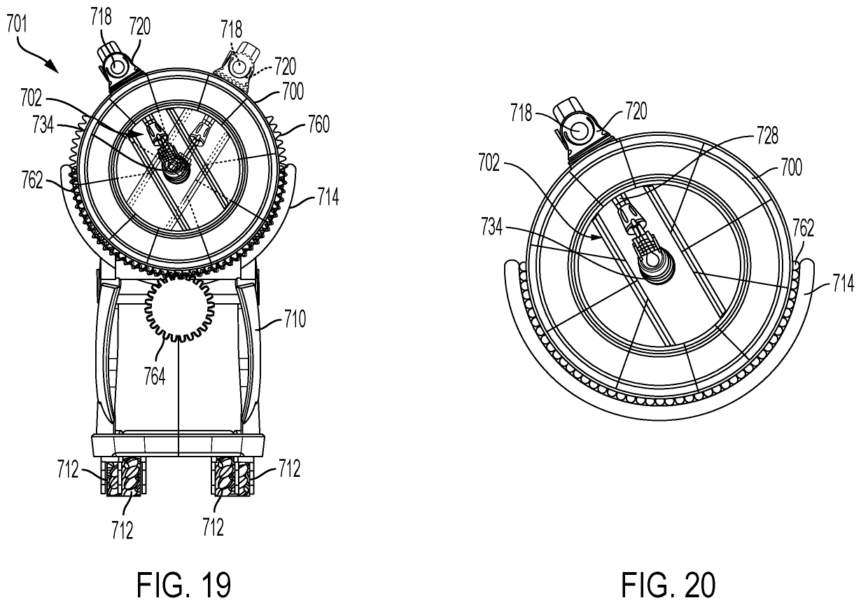

Referring primarily to FIGS. 19-21, the cart 710 can include a driving actuator 766. The driving actuator 766 can be constructed of a material or materials that do not or minimally interfere with MRI (e.g., the driver actuator 766 can be MR-safe and/or MR-compatible). The driving actuator 766 can include any type of actuator, such as, for example, a pneumatic actuator, electric actuator, or a hydraulic actuator. The driving actuator 766 can generate torque to rotate the MRI scanner 700. The cart 710 can further include a driving modality 764 (e.g. a gear) that is operatively coupled to the driving actuator 766. The driving actuator 766 and driving modality 764 can be constructed of a material or materials that do not or minimally interfere with MRI (e.g., the driving actuator 766 and the driving modality 764 are MR-safe and/or MR-compatible). Further, the MRI scanner 700 can include driven modality 760 (e.g. gear teeth). The driven modality 760 can be constructed of a material or materials that do not or minimally interfere with MRI (e.g., the driven modality 760 is MR-safe and/or MR-compatible). The driving modality 764 and the driven modality 760 can be operatively coupled such that actuating the driver actuator 766 causes the MR scanner 700 to rotate about the axis 780 (FIG. 17). The driving modality 764 and the driven modality 760 can include any type and combinations of components for transmitting torque from the driving actuator 766 to rotate the MR scanner 700. For example, the driving modality 764 and the driven modality 760 can include any combination and/or type of gears, pulleys, sprockets, etc. The driving modality 764 and/or the driving actuator 766 can be positioned inside or outside of the cart 710 to efficiently transmit power to the driven element 760. The frame of the MRI scanner 700 can include curved rails 714 supported by frictionless rolling elements 762 to enable safe and smooth rotation of the MRI scanner 700. For example, the driving actuator 766 can cause the driving modality 764 to drive the driven modality 760 to rotate the MRI scanner 700 along the rails 714.

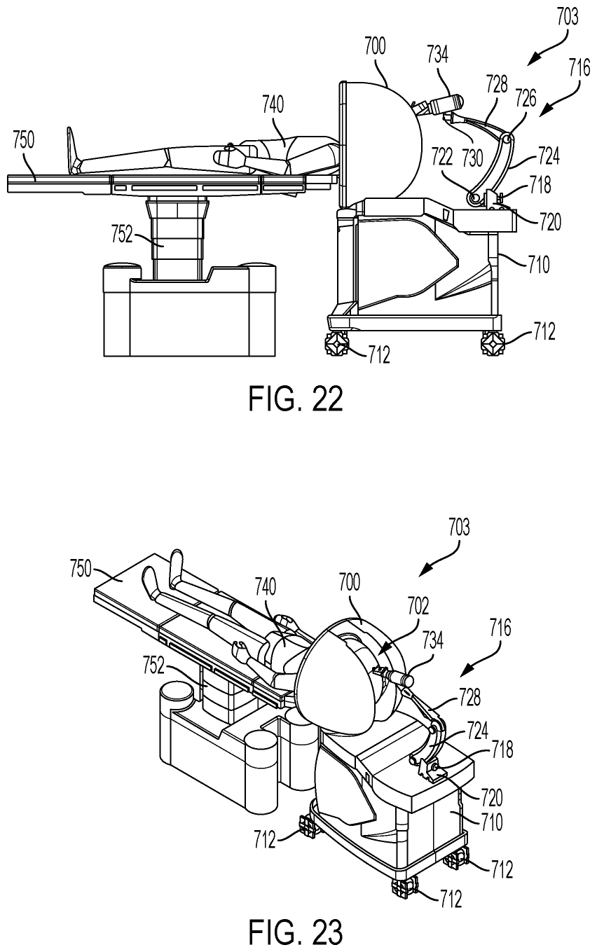

Referring still primarily to FIGS. 19, 20, and 21, although the adjustable arm 716 is depicted as being supported by the MR scanner 700, other support configurations for the adjustable arm 716 are contemplated by the present disclosure. For example, the adjustable arm 716 may be mounted on or otherwise supported by the cart 710, as described below with respect to FIGS. 22 and 23.

FIGS. 22 and 23 illustrate an MRI-guided surgical robotic system 703 (the system 703), according to at least one aspect of the present disclosure. The system 703 is similar in many aspects to the system 701 described above, with corresponding reference numerals indicating similar components. In the non-limiting aspect of FIGS. 22 and 23, the arm 716 is mounted or otherwise supported by the cart 710. Thus, the MRI scanner 700 and the arm 716 (and thus the surgical robot 734) can move independently. Furthermore, in some aspects, compared to the system 701, the system 703 can provide more stability and stiffness for supporting the arm 716 and/or the surgical robot 734. Accordingly, the system 703 may be configured to, in some aspects, provide increased force exertion and higher positioning accuracy of the surgical robot 734 compared to the system 701 (e.g., based on the system 703 having a stationary rigid base (e.g. the cart 710)). The system 703 can provide a similar range of arm 716 motion and workspace footprint.

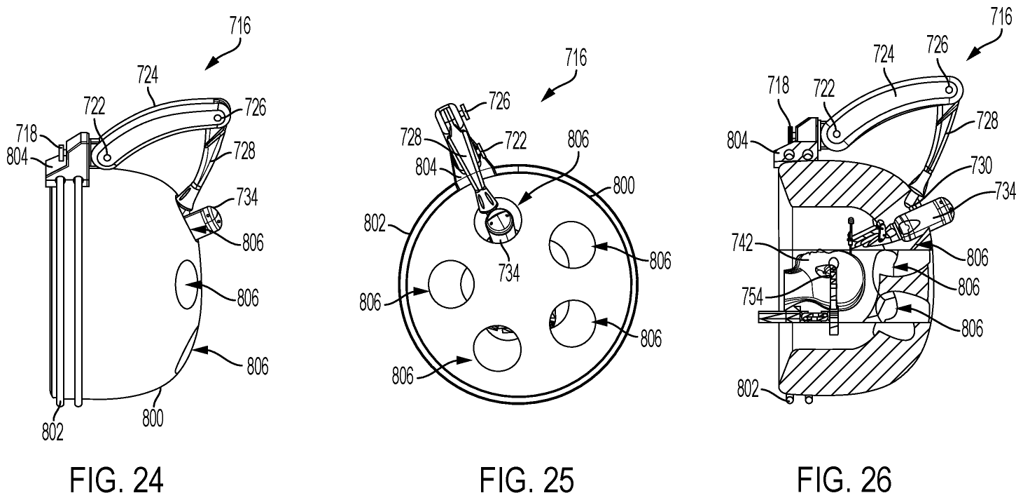

FIGS. 24-26 illustrate an MRI scanner 800, according to at least one aspect of the present disclosure. The MRI scanner 800 is similar in many aspects to the MRI scanner 700 described above, with corresponding reference numerals indicating similar components. The MRI scanner 800 can be used with any of the systems described herein (e.g., system 701, 703).

In the non-limiting aspects of FIGS. 24, 25 and 26, the MRI scanner 800 includes a guide 802 (e.g., one or more rails). The guide 802 can extend around a circumference of the MRI scanner 800. The adjustable arm 716 can be mounted or otherwise supported by a carriage 804. The carriage 804 can be operatively coupled to the guide 802 to enable the arm 716 to move about the circumference of the MRI scanner 800. For example, by sliding or otherwise moving the carriage 804 about the guide 802, the arm 716 and/or the surgical robot 734 can be oriented at any angle relative to a vertical or horizontal plane of the MRI scanner 800. In some aspects, the carriage 804 can be locked in place relative to the guide 802 once a desired position is achieved to prevent movement of the carriage 804. In various aspects, a motor inside of the carriage 804 is configured to move the carriage 804 along the guide 802.

Referring still to FIGS. 24-26, the MRI scanner 800 can include one or more than one opening 806. The one or more than one opening 806 can include any type and combination of openings (e.g., circular openings, slots, rectangular openings, etc.). The one or more than one opening 806 can be configured in any pattern on the MRI scanner 800. For example, the one or more than one opening 806 can be distributed circumferentially in the form of circle, ellipse, arc slot or any other form factors that open a corridor to enable the surgical robot 734 to pass through the MRI scanner 800 and access the head 742 of the patient 740.

In at least one aspect, the arm 716 has at least two positional degrees of freedom (DOFs) and at least two DOFs of force feedback. The at least two DOFs allow the arm 716 to reach and position the surgical robot 734 through an opening 806 in the dome-shaped housing of the MRI scanner 800. For example, the arm 716 and surgical robot 734 can be positioned in and out of an opening 806 in the dome-shaped housing of the MRI scanner 800.

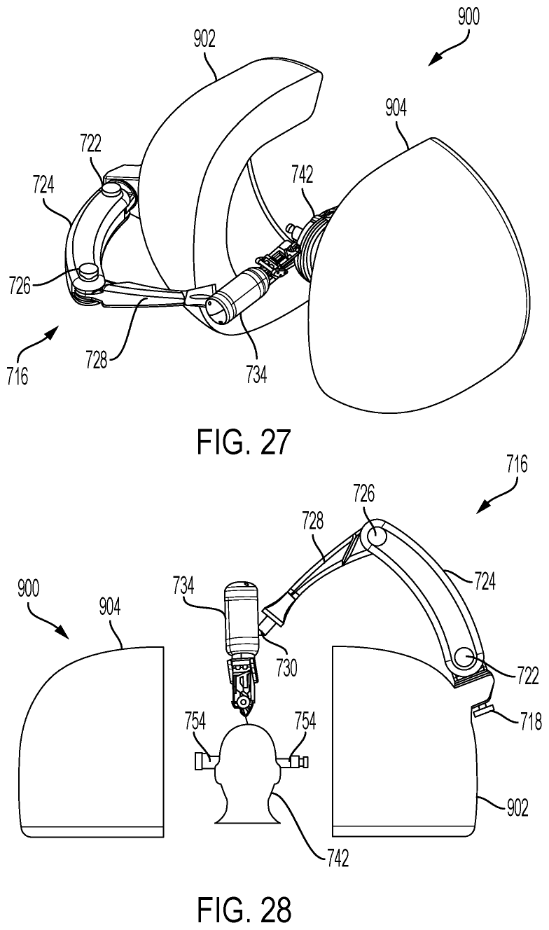

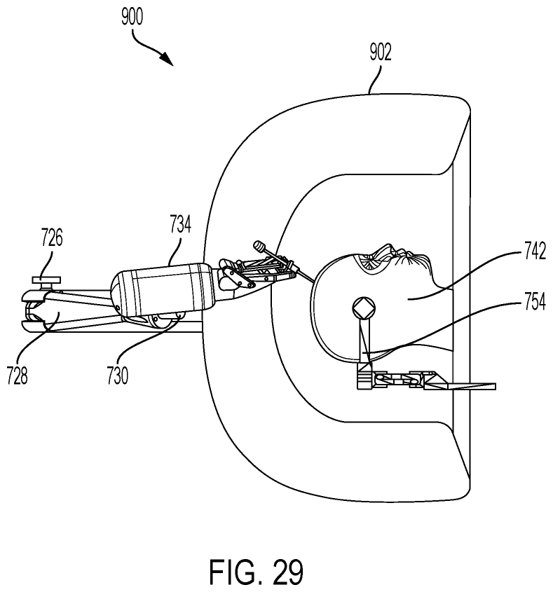

FIGS. 27-29 illustrate an MRI scanner 900, according to at least one aspect of the present disclosure. The MRI scanner 900 is similar in many aspects to the MRI scanner 700 and the MRI scanner 800 described above, with corresponding reference numerals indicating similar components. The MRI scanner 900 can be used with any of the systems described herein (e.g., system 701, 703).

In the non-limiting aspects of FIGS. 27-29, the MRI scanner 900 includes two elliptical splitable segments, which are designated as segment 902 and segment 904. A plane defining the split of the segment 902 and the segment 904 can be vertical, as depicted in FIGS. 27, 28, and 29, or can be any other orientation (e.g., horizontal, any angle relative to horizontal or vertical). The arm 716 can be mounted or otherwise supported by either the segment 902 or the segment 904. Further, the arm 716 can be mounted or otherwise supported by either segment 902 or 904 at any an angle relative to the vertical or horizontal plane of the MRI scanner 900 (e.g., an angle that provides a maximum reachable workspace for the surgical robot 734 based on the motion allowable by the shoulder 722 and elbow 726 joints of the arm 716). Additionally, in some aspects, the arm 716 can be mounted to a cart 710 as described in regard to FIGS. 22 and 23. In some aspects, the adjustable gap between the segment 902 and the segment 904 can be closed during imaging and can be open during intervention of the surgical robot 734. In an alternative aspect, the adjustable gap between the segment 902 and the segment 904 can be open during imaging and during the intervention of the surgical robot 734. Furthermore, the adjustable gap between the segment 902 and the segment 904 can be open as the patient 740 and the MRI scanner 900 are positioned relative to one another.

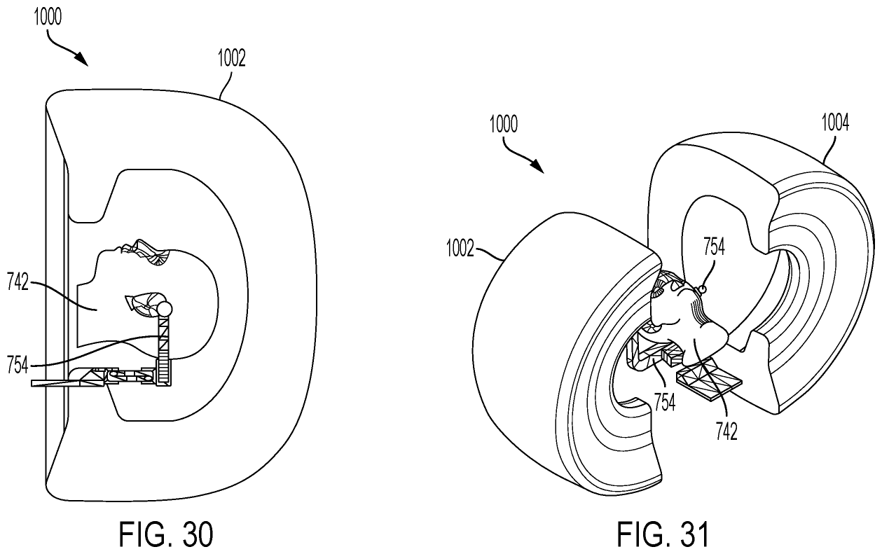

FIGS. 30 and 31 illustrate an MRI scanner 1000, according to at least one aspect of the present disclosure. The MRI scanner 1000 is similar in many aspects to the MRI scanner 700, the MRI scanner 800, and the MRI scanner 900 described above, with corresponding reference numerals indicating similar components. The MRI scanner 1000 can be used with any of the systems described herein (e.g., system 701, 703).

In the non-limiting aspects of FIGS. 30 and 31, the MRI scanner 1000 includes two divisible narrow-neck segments, which are designated as segment 1002 and segment 1004. The plane defining the split of the segment 1002 and the segment 1004 can be vertical, as depicted in FIGS. 30 and 31, or can be any other orientation (e.g., horizontal, any angle relative to horizontal or vertical). The arm 716 can be mounted or otherwise supported by either the segment 1002 or the segment 1004. Further, the arm 716 can be mounted or otherwise supported by either segment 1002 or 1004 at any an angle relative to the vertical or horizontal plane of the MRI scanner 1000 (e.g., an angle that provides a maximum reachable workspace for the surgical robot 734 based on the motion allowable by the shoulder 722 and elbow 726 joints of the arm 716). Additionally, in some aspects, the arm 716 can be mounted to a cart 710 as described in regard to FIGS. 22 and 23. In some aspects, the adjustable gap between the segment 1002 and the segment 1004 can be closed during imaging and can be open during intervention of the surgical robot 734. In an alternative aspect, the adjustable gap between the segment 1002 and the segment 1004 can be open during imaging and during the intervention of the surgical robot 734. Furthermore, the adjustable gap between the segment 1002 and the segment 1004 can be open as the patient 740 and the MRI scanner 1000 are positioned relative to one another.

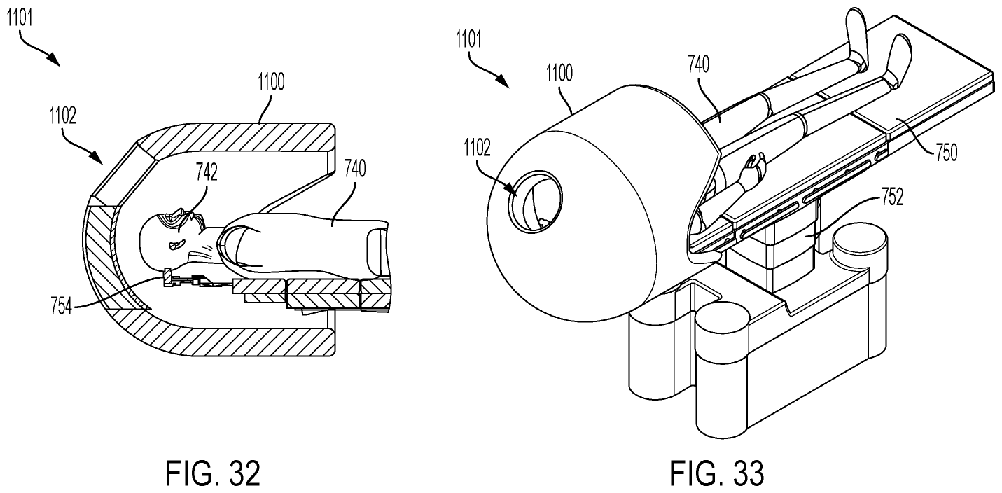

FIGS. 32 and 33 illustrate an MRI-guided surgical robotic system 1101 (the system 1101), according to at least one aspect of the present disclosure. The system 1101 is similar in many aspects to the system 701 and the system 703 described above, with corresponding reference numerals indicating similar components. The system 1101 can include an MRI scanner 1100. The MRI scanner 1100 is similar in many aspects to the MRI scanner 700, the MRI scanner 800, the MRI scanner 900, and the MRI scanner 1000 described above, with corresponding reference numerals indicating similar components. The MRI scanner 1100 can be used with any of the systems described herein (e.g., system 701, 703, 1101).

As depicted by FIGS. 32 and 33, the MRI scanner 1100 includes a long bore to cover longer target limb extremities of the patent 740. Further, the scanner 1100 includes at least one opening 1102 for surgical-related access (e.g., access by the surgical robot 734 and/or by surgical staff to the lesion, entry points, fiducial markers, intubation or draping). In at least one aspect, arm 716 can be mounted or otherwise supported by the scanner 1100 as described above in regard to FIGS. 15-21 and 24-29. Additionally, in some aspects, the arm 716 can be mounted to a cart 710 as described in regard to FIGS. 22 and 23.

In at least one aspect, the patient table 750 can move a patient 740 relative to the MRI scanner 1100 to position the patient 740 within the MRI scanner 1100. In an alternative aspect, the MRI scanner 1100 is moved relative to the table 750 to position the patient 740 within the MRI scanner 1100. In at least one aspect, the MRI scanner 1100 can be mechanically coupled to a cart (e.g. cart 710) to allow a clinician to move the MRI scanner 1100 relative to the table 750 to position the patient 740 within the MRI scanner. In an alternative aspect, the MRI scanner 1100 is mechanically attached to the table 750 (e.g. by rails) to allow the table and/or the MRI scanner 1100 to move to position the patient 740 within the MRI scanner.

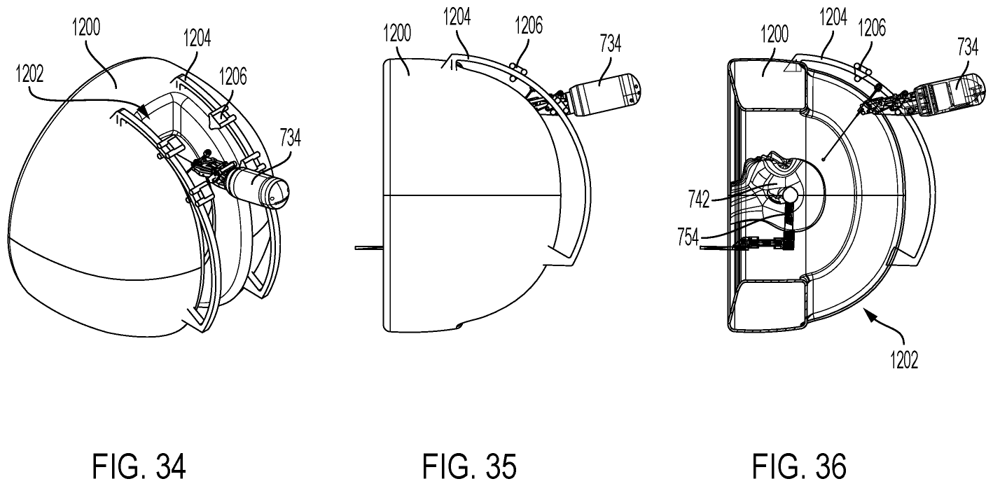

FIGS. 34-36 illustrate an MRI scanner 1200, according to at least one aspect of the present disclosure. The MRI scanner 1200 is similar in many aspects to the MRI scanner 700, the MRI scanner 800, the MRI scanner 900, the MRI scanner 1000, and the MRI scanner 1100 described above, with corresponding reference numerals indicating similar components. The MRI scanner 1200 can be used with any of the systems described herein (e.g., system 701, 703, 1101).

In the non-limiting aspects of FIGS. 34-36, the MRI scanner 1200 includes an opening 1202 (e.g., a slot) and a guide 1204 (e.g., one or more than one rail) positioned along the opening 1202. The surgical robot 734 can be mounted or otherwise supported by a carriage 1206. The carriage 1206 can be operatively coupled to the guide 1204 to enable the surgical robot 734 to move relative to the opening 1202. For example, by sliding or otherwise moving the carriage 1206 along the guide 1204, the surgical robot 734 can be oriented at any position relative to the opening 1202. In various aspects, a motor inside of the carriage 1206 is configured to move the carriage 1206 along the guide 1204. In some aspects, the carriage 1206 can be locked in place relative to the guide 1204 once a desired position is achieved to prevent movement of the carriage 1206. Thus, a position of the surgical robot 734 can be adjusted and locked at a specific angle relative to the desired longitudinal and latitudinal coordinate.

Any aspect of the various systems 701, 703, 1101 and MRI scanners 700, 800, 900, 1000, 1100, 1200 described herein can be applied to a different one of the various systems 701, 703, 1101 and MRI scanners 700, 800, 900, 1000, 1100, 1200.

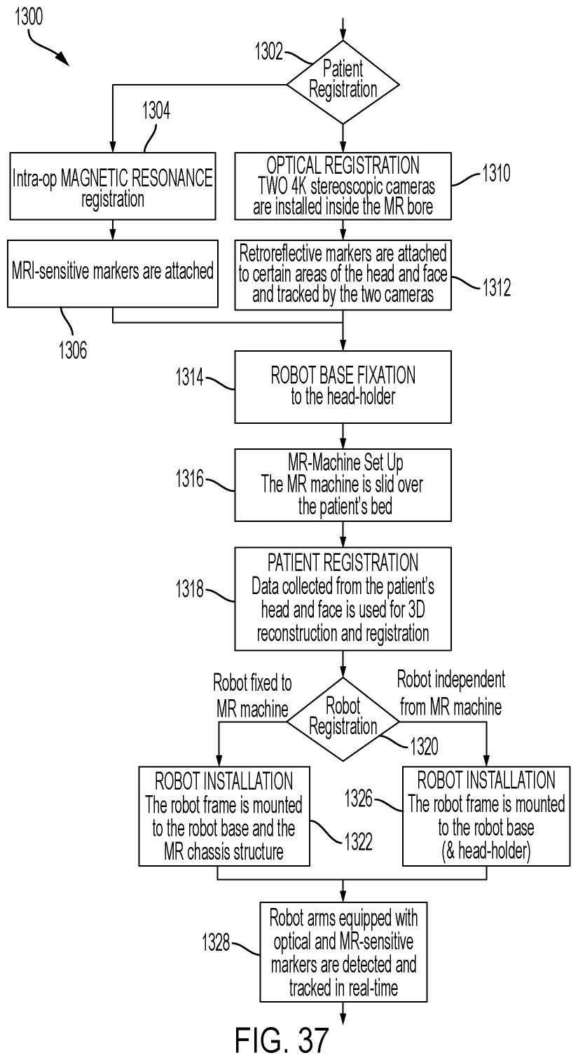

FIG. 37 depicts a method 1300 for operating an MRI-guided surgical robotic system, according to at least one non-limiting aspect of the present disclosure. Although the method 1300 is described below as being implemented by various components of the system 701, the method 1300 can be implemented by any of the systems described herein, such as the system 703 or system 1101 and can be used with any of the MRI scanners 700, 800, 900, 1000, 1100, 1200.

Referring primarily to FIG. 37, according to various aspects of the method 1300, a decision can be made as to whether to register 1302 a patient 740. Basic pre-op procedures may be performed. For example, the head 742 of the patient 740 can be fixed via the head fixation device 754. The patient 740 may be draped. The cart 710 may be moved proximate to the patient bed 750.

Referring still primarily to FIG. 37, according to the method 1300, in one aspect, intra-operative MR registration may be performed 1304 using the MRI scanner 700. Intra-operative MR registration is a form of touchless tracking. Further, MRI-sensitive markers may be attached 1306 (e.g., to the head 742 of the patient 740 and/or the surgical robot 734). The MRI scanner 700 can be used to collect data of the MRI-sensitive markers. The data can be analyzed to determine the position of the MRI-sensitive markers allowing the position of the patient and/or the surgical robot 734 to be tracked in space.

Referring still primarily to FIG. 37, according to the method 1300, in an additional or alternative aspect, optical registration may be performed 1310. Optical registration is a form of touchless tracking. For example, a stereoscopic camera (e.g., two 4K stereoscopic cameras) may be placed inside a bore of the MR scanner 700. Furthermore, retroreflective markers may be attached 1312 to the head 742 of the patient and/or the surgical robot 734. The stereoscopic camera may record image data of the retroreflective markers. Visual analysis of the image data can be performed to determine the position of the retroreflective markers allowing the position of the patient and/or the surgical robot 734 to be tracked in space.

In some alternative aspects, touch-based tracking may be performed to track the position of the patient in space. Touch-based tracking can limit the workspace of the surgical robot 734. For example, touch-based tracking can take up space inside of the MRI scanner 700, which provides the surgical robot 734 with less room to be oriented within the MRI scanner 700.

Referring still primarily to FIG. 37, according to the method 1300, the surgical robot 734 may be fixed 1314 to components of the arm 716 and/or the base stand 720. The cart 710 may be moved 1316 proximate to the patient bed 750. For example, the cart 710 may be positioned such that the head 742 of the patient 740 is inside the bore of the MRI scanner 700 (e.g., as depicted by FIG. 11). Furthermore, the patient 740 can be registered 1318. For example, using the MRI scanner 700, data can be collected related to the head 742 of the patient 740. The data can be used for 3D reconstruction and registration. In at least one aspect, the data includes data collected with the two 4K stereoscopic cameras. In an additional or alternative aspect, the data includes data from the MRI-sensitive markers.

Referring still primarily to FIG. 37, according to the method 1300, the surgical robot 734 can be registered 1320. Registration 1320 can be based on whether the surgical robot 734 is supported by the MRI scanner 700 (e.g., as depicted by FIGS. 9-21) or the surgical robot 734 is supported by the cart 710 (e.g., as depicted by FIGS. 22-23).

Referring still primarily to FIG. 37, according to the method 1300, in aspects where the surgical robot 734 is supported by the MRI scanner 700, the surgical robot 734 can be installed 1322. Installation 1322 of the surgical robot 734 can include coupling the surgical robot 734 (e.g. the arm 716 and/or the base stand 720) to the MRI scanner 700.

Referring still primarily to FIG. 37, according to the method 1300, in aspects where the surgical robot 734 is supported by the cart 710, the surgical robot 734 can be installed 1326. Installation 1326 can include coupling the surgical robot 734 (e.g. the arm 716 and/or the base stand 720) to the cart 710.

In some aspects, the arm 716 and/or the surgical robot 734 may be equipped with optical and/or MRI-sensitive markers for registration 1328 similar to the registration performed on the patient. The position of the optical and/or MRI-sensitive markers may be tracked by the MRI system in real time during a surgical procedure. This process allows the position of the arm 716 and surgical robot 734 to be tracked in space. In at least one aspect, once the patient 740 and the surgical robot 734 are registered, then the position of the surgical robot 734 can be tracked relative to the position of the patient 740.

In some aspects, registration 1318, 1328 of the surgical robot 734 and the patient 740 is performed by the fusion of images acquired through a set of stereoscopic cameras placed inside the bore of the MRI scanner 700 and images taken via intra-operative MRI. In some aspects, an optimized custom design of the bore of the MRI scanner 700 and manipulability of the surgical robot 734 can allow for easy access to the head 742 of the patient 740 during a surgical procedure to perform validation and/or for emergency interventions.

The surgical robot 734 and the MRI scanner 700 can be cooperatively utilized to perform a surgical procedure. For example, the MRI scanner 700 can be used to generate an image that guides the surgical robot 734 during a surgical procedure. In at least one aspect, the surgical robot 734 is within the MRI scanner 700 during the data collection to generate an image. For example, the surgical robot 734 can be operated during an MRI scan. In an alternative aspect, the surgical robot 734 is removed from within the MRI scanner 700 during the data collection to generate an image.

While FIGS. 9-37 discuss various MRI scanners 700, 800, 900, 1000, 1100, 1200 in relation to scanning a patient's head, the MRI scanners 700, 800, 900, 1000, 1100, 1200 can be used to generate an image of other extremities (e.g. upper or lower limbs) positioned within the field of view of the respective scanner.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a control circuit computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A system, comprising:
a magnetic resonance imaging (MRI) scanner comprising a housing forming a dome, wherein a region of interest is defined within the dome, wherein the housing comprises a permanent magnet array forming a modified Halbach array, wherein the MRI scanner is to generate an image of the region of interest, and wherein an opening is defined through the housing into the region of interest; and
a surgical robot comprising a robotic arm, wherein the robotic arm is mounted to the MRI scanner, and wherein the robotic arm is dimensioned to pass through the opening in the housing.

2. The system of claim 1, wherein the dome comprises a curved wall and a rim, and wherein the opening is defined through the curved wall.

3. The system of claim 1, wherein the housing defines a bore having a longitudinal axis, and wherein the permanent magnet array is to generate a B₀ magnetic field that is aligned with the longitudinal axis.

4. The system of claim 3, wherein the B₀ magnetic field is less than 1.0 T.

5. The system of claim 3, wherein the B₀ magnetic field is less than 0.1 T.

6. The system of claim 3, further comprising a mobile cart, wherein the MRI scanner is rotatably mounted to the mobile cart.

7. The system of claim 6, wherein the opening defines a slot, and wherein rotating the MRI scanner adjusts an angle of the slot.

8. The system of claim 1, further comprising a wheeled cart, wherein the MRI scanner and the surgical robot are mounted to the wheeled cart.

9. The system of claim 8, wherein the wheeled cart comprises a rotary actuator operatively coupled to the MRI scanner, and wherein actuating the rotary actuator rotates the housing about a longitudinal axis.

10. The system of claim 9, wherein the wheeled cart is releasably attachable to a patient table.

11. The system of claim 10, wherein the patient table comprises a fixation device for holding a head of a patient, and where the MRI scanner is positionable to surround the fixation device and the head of the patient.

12. The system of claim 1, wherein the housing further comprises a guide, wherein the surgical robot comprises a carriage operatively coupled to the guide, and wherein the carriage is slidable along the guide to adjust a position of the surgical robot.

13. The system of claim 1, wherein the MRI scanner is to generate the image in real time while the surgical robot is performing a surgical procedure.

14. The system of claim 1, further comprising a surgical end effector extending from the robotic arm, wherein the robotic arm comprises at least two degrees of freedom to selectively position the surgical end effector through the opening into the region of interest.

15. A method of performing a surgical procedure with an MRI-guided surgical robotic system, the method comprising:
positioning a patient on a patient table, wherein the patient table comprises a fixation device;
fixing a head of the patient to the fixation device;
moving a cart supporting an MRI scanner proximate to the patient table, wherein the head of the patient is positioned within a dome-shaped housing of the MRI scanner, wherein the dome-shaped housing defines an imaging region;
positioning a surgical robot relative to the MRI scanner with a robotic arm;
co-registering the MRI scanner and the robotic arm with the head; and
cooperatively utilizing the surgical robot and the MRI scanner to perform the surgical procedure.

16. The method of claim 15, wherein the dome-shaped housing further comprises an opening for accessing the head of the patient, and wherein the method further comprises moving the robotic arm through the opening to position a surgical end effector attached to the robotic arm within imaging region.

17. The method of claim 15, wherein the method further comprising rotating the dome-shaped housing to position the opening in a first configuration.

18. The method of claim 17, further comprising rotating the dome-shaped housing to position the opening in a second configuration.

19. The method of claim 15, further comprising using the surgical robot and the MRI scanner simultaneously.

20. The method of claim 15, further comprising operating the surgical robot during an active MRI scan.
